(19) United States
(12) Patent Application Publication
(10) Pub. No.: US 2008/0318326 A1
(43) Pub. Date: Dec. 25, 2008

(54) Method of Manufacture

(75) Inventors: Hong Thai Phung, Palmerston North (NZ); James Dunlop, Palmerston North (NZ); Julie Eleanor Dalziel, Palmerston North (NZ); Yan-Li Zhang, Palmerston North (NZ)

Correspondence Address: SUGHRUE MION, PLLC, 2100 PENNSYLVANIA AVENUE, N.W., SUITE 800, WASHINGTON, DC, 20037, US

(73) Assignee: AGRESEARCH LIMITED, Hamilton (NZ)

(21) Appl. No.: 12/066,005

(22) PCT Filed: Sep. 1, 2006
(86) PCT No.: PCT/NZ2006/000228

§ 371 Date: Aug. 5, 2008

(30) Foreign Application Priority Data
Sep. 7, 2005 (NZ) 542286
Jun. 23, 2006 (NZ) 548138

(51) Int. Cl. G01N 33/68; B05D 3/00

(52) U.S. Cl. 436/86; 427/322

(57) ABSTRACT

The invention relates to methods of preparing a bilayer lipid membrane on a support matrix as well as a matrix preloaded with a protein and methods to achieve same. Steps used include hydration, pore infiltration, application of bilayer lipid membrane forming solutions and application of protein containing solutions. The methods and matrix produced are able to form and maintain a stable membrane. A further advantage is that the matrix may be pre-loaded with protein and then stored for use at a later date effectively stabilising the protein for later use.

### TECHNICAL FIELD

This invention relates to a method of manufacturing bilayer lipid membranes and preloading functional proteins into a support matrix.

### BACKGROUND ART

Over recent years there has been a marked increase in the interest and research of biotechnologies. Areas of interest include the use of biosensors in medicine, environmental monitoring, biosecurity, and drug discovery. They offer a wide range of benefits including selectivity and sensitivity in detecting target compounds.

Biotechnologies are based on the in vitro application of biological processes and molecules. These molecules, often based on proteins, can be genetically modified to allow direction to the specific characteristics desirable for a particular situation.

However, there are significant challenges relating to the preparation of this biotechnology. These include preparing devices which are suitably robust to allow practical applications and devices to be developed. A biotechnology device would be robust enough to withstand a variety of conditions for an extended period of time.

Existing methods to construct biological membranes utilise phospholipid bilayers. These bilayer membranes are comprised of single sheets of phospholipids which are two molecules thick. The molecules are aligned in a sheet-like arrangement so that the hydrophilic phosphate head residues are at the surface of the bilayer with the hydrophobic lipid tails facing towards the centre. This creates a bilayer approximately 4 nanometres thick.

Throughout the bilayer membrane sheet proteins are incorporated into the sheet. These proteins are responsible for a range of cellular functions that include recognition, signalling, energy transduction, the development of energy gradients, discrimination, filtering, concentration of molecules/ions, and the transport of nutrients and metabolites.

It is the efficiency, sensitivity and selectivity of these membrane proteins which has the most potential in the development of a new biotechnology specifically for use as biosensors that employ membrane proteins such as receptors and/or ion channels.

A disadvantage of bilayer membranes is that they are very thin meaning they are extremely fragile and require careful handling. To improve their stability in biotechnologies it is necessary to support them using some form of substrate matrix.

Various supports have been developed for phospholipid molecules including metal surfaces, hydro-gels, derivatised gold, and silicate sol-gels.

Bilayers supported on metal surfaces tend to be more robust than other supports. But, as metals are impermeable to water, they are not suitable as supports for membrane proteins whose function requires movement of ions or molecules through the membrane.

Hydro-gels support bilayers and also allow the function of membrane proteins involved in the translocation of ions and molecules. However, bilayers supported on hydro-gels still have low stability.

Other solid, porous structures have been used to support bilayer membranes. These include glass fibre surfaces, triacetyl cellulose, nitrocellulose, polycarbonate, alumina, and millipore filters made from polytetrafluoroethylene (PTFE).

The results of studies using these various solid, porous structures to support bilayer membranes have had limited success in achieving formation of a bilayer lipid membrane on the surface.

A study by Thomson, Lennox and McClelland in 1982 investigated the potential to support a bilayer lipid membrane on PTFE filters, cellulose acetate surfaces, and polycarbonate filters. The study attempted to form a bilayer lipid membrane by applying a phospholipid solution to the dry support matrices and then placing them in contact with an aqueous solution. The studies reported that PTFE was an inferior support material compared to the polycarbonate filters. It is considered by the inventors that it is unclear from the results of the study whether a bilayer lipid membrane was formed on PTFE. The majority of successful published results in the study were achieved using a polycarbonate support with only one result directed to formation of a bilayer lipid membrane on PTFE. There is no replication or errors associated with this one result. Further, the results are compared with interpolated data from another study. This leads to assumptions being made to explain the observations. Because of these shortcomings, there are possible valid explanations for the result other than the formation of a bilayer lipid membrane.

The authors of the study offer evidence for the formation of a bilayer lipid membrane on polycarbonate filters such as a plot of current versus voltage that is slightly curvilinear and attribute this to a valid result for all three types of filter used to support matrices in the study. However, the problem is that the results could be explained in a variety of ways e.g. an application of Ohms law and the use of a silver/silver chloride electrode.

Regardless of whether a bilayer lipid membrane was formed in this study, there is no definitive evidence that the alleged bilayer lipid membranes were shown to be suitable to support functions such as ion channel conduction. Other areas left undetermined include: (a) whether or not the membrane formed was capable of withstanding a variety of experimental conditions, (b) was the membrane stable enough to allow the easy removal of the bilayer lipid membrane and the subsequent formation of a new bilayer lipid membrane with functional ion channels, or (c), could the membrane be preloaded with ion channel membrane proteins prior to the formation of the bilayer lipid membrane?

It is the inventors' hypothesis that the lack of evidence for the formation of a bilayer lipid membrane may be due to the method by which the supposed phospholipid bilayer was formed.

There have been several studies undertaken by different researchers where a bilayer lipid membrane has been reported to have formed on porous materials i.e. a filter. These studies demonstrated the successful incorporation of a functional non-protein, ion channel proteins, or integral membrane proteins (similar to ion channel proteins), into the formed bilayer lipid membrane. However, these studies used small peptide ion channel molecules (most notably gramicidin and alamethicin) that are not proteins. These peptides are very different to the physiologically important ion channel proteins, are much smaller in molecular size, and have a greater tolerance to storage and experimental conditions than in situ ion channel proteins.

Other studies have used glass fibre support matrices, forming a bilayer lipid membrane on the surface of aqueous solutions which was then transferred to the filter by a technique known as folding. Folding is known to one skilled in the art as passing the support material through a monolayer of phospholipids on the surface of the solution.

Studies using glass fibre support matrices have only utilised proteins such as enzymes. These are significantly different from more complex proteins such as ion channel proteins and integral membrane proteins, hence similar results would not be an immediate assumption.

To the inventors' knowledge, there is no reference in studies that use glass fibre support matrices to the longevity, reusability, or preloading of the support matrix. A study by Mountz and Tien, 1978 reported a system involving porous polycarbonate support matrices. In these experiments they applied a mixture of phospholipid solutions with an extract of chloroplasts to a dry support which was then placed in aqueous solution. This method utilises a hydration step in which an aqueous solution is applied to the support matrix after the application of the phospholipid containing solution. In addition, this study used a solution which contained a combination of many different types of proteins. Therefore, it is difficult to determine whether this study provides a robust and durable planar bilayer lipid membrane which allows the normal function of complete, integral membrane proteins as ion channels.

Several studies have used lipid impregnated PTFE filters and subsequently used a membrane protein (rhodopsin, a G-protein coupled receptor), to form the ion channel. This membrane protein has structural features and requirements similar to biological ion channels. The filters are prepared by infiltrating them with lipid solutions before they are placed in contact with water or aqueous solutions. The authors of the studies do not claim or demonstrate that a bilayer lipid membrane has been formed. The authors acknowledge the protein is contained in membrane vesicles that are “associated” with the impregnated filter.

It should be appreciated from the above that it would be advantageous to have a method which allowed the formation of a phospholipid bilayer lipid membrane supported by a matrix which may be:
1. More robust and durable than a standard planar bilayer lipid membrane;
2. Allows for the normal function of complete integral membrane proteins as functional proteins;
3. Was amenable to the introduction of functional proteins through fusion of proteoliposomes containing the proteins;
4. Was stable but still allowed the easy removal of the bilayer lipid membranes and the subsequent formation of a new bilayer lipid membrane in combination with functional proteins.
5. Allowed for the formation of support surfaces preloaded with functional proteins prior to formation of the bilayer lipid membrane.

It is therefore an object of the present invention to address the foregoing problems or at least to provide the public with a useful choice.

All references, including any patents or patent applications cited in this specification are hereby incorporated by reference. Priority applications NZ 542286 and NZ 548138 are also incorporated herein by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF THE INVENTION

According to one aspect of the present invention there is provided a method of preparing a bilayer lipid membrane, including the steps of:

a) preparing a support matrix;
b) hydrating the support matrix using a hydration solution; and,
c) applying a lipid containing solution to the hydrated support matrix which forms a bilayer lipid membrane on the support matrix.

According to a further aspect of the present invention there is provided a method of preparing a bilayer lipid membrane loaded with at least one protein, including the steps of:

a) preparing a support matrix;
b) hydrating the support matrix using a hydration solution;
c) applying a lipid containing solution to the hydrated support matrix which forms a bilayer lipid membrane on the support matrix; and,
d) applying a protein containing solution to the hydrated support matrix of step (c).

According to a further aspect of the present invention there is provided a method of preparing a pre-loaded protein containing support matrix, including the steps of:

a) preparing a support matrix;
b) hydrating the support matrix using a hydration solution;
c) applying a protein containing aqueous solution to the hydrated support matrix of step (b).

In a preferred embodiment based on the method above, the solution used completes both steps (b) and (c) simultaneously.

According to a further aspect of the present invention there is provided a method of preparing a pre-loaded protein containing support matrix, including the steps of:

a) preparing a support matrix;
b) hydrating the support matrix using a hydration solution;
c) applying a lipid containing solution to the hydrated support matrix which forms a bilayer lipid membrane on the support matrix;
d) applying a protein containing aqueous solution to the hydrated support matrix of step (c); and;
e) washing the bilayer lipid membrane from the support matrix.

According to a further aspect of the present invention there is provided a support matrix including at least one protein.

The inventors have utilised support matrix, membrane and protein technologies to produce a matrix assembly useful for producing a bilayer lipid membrane, stabilising proteins, and for measuring protein activity.

In the present invention the term ‘support matrix’ refers to a material which is capable of supporting a bilayer lipid membrane and in which functional protein activity can be observed.

In one example, ion channel activity represents one indicator of protein activity.

In a preferred embodiment, the support matrix materials are characterised by high hydrophobicity and high resistance to flow through pores.

Preferably, the hydrophobicity of the preferred material corresponds to a contact angle greater than 50°.

Preferably, the bubble point of the preferred material is greater than 0.20 corresponding to a higher resistance to the flow of fluids through the pores (and thereby more irregular/tortuous pore shapes).

In a preferred embodiment, the support matrix is PTFE. However, this should not be seen as limiting as other materials with similar properties of hydrophobicity and/or pore shape may be used including, but not limited to, nylon.

In preferred embodiments, the support matrix material is attached to a fixture that holds the matrix in place and directs flow of liquid through the matrix.

In one embodiment the fixture is a cube shaped polystyrene cuvette where the support matrix seals at least one aperture in the cuvette such that liquid must pass through the aperture and support matrix.

The inventors also envisage a fixture including a dual chamber apparatus with a support matrix located between the chambers. The chambers would be constructed from water tight materials and have corresponding apertures on one face of each chamber. The support matrix would be inserted between the apertures and an alignment system used to bring the chambers into contact and secure them such that they form a seal and hold the support matrix in place.

Another embodiment envisaged for fixing the support matrix in place is a tube arrangement where a disk of the support matrix material is folded around one end of a piece of water tight tubing. A ring of a second piece of tubing of diameter greater than the first is placed over the support matrix such that it forms a water tight seal and holds the support matrix in place.

In a further embodiment of the present invention the support matrix may be a multi-well filter plate. This is a plate which has two or more reservoirs which can each secure an individual support matrix. This configuration for the support matrix is beneficial because it provides a user with flexibility in how they use the present invention. For example, different functional proteins may be used with each discrete support matrix.

Alternatively, it may be possible to have different support matrices within each reservoir of the multi-well filter plate, or to form bilayer lipid membranes from different lipid containing solutions in each reservoir.

The above examples should not be seen as limiting as it will be appreciated that a key aspect is directing flow of liquid across the matrix and the fixture or holder for this matrix can take many forms.

A critical step found by the inventors is the need for correct and thorough hydration of the support matrix. Hydration dramatically improves the success in forming a bilayer lipid membrane.

In a preferred embodiment, hydration may be completed using an electrolyte solution. Preferably, the solution is an aqueous electrolyte solution prepared with water as the principal solvent along with solutes that dissociate into ions, i.e. electrically charged particles.

Hydrating the support matrix is critical as it results in a high success rate for forming a bilayer lipid membrane (more than has previously been achieved in the prior art). It is the inventors' experience that this overcomes problems with previous studies attempting to form bilayer lipid membranes, particularly on PTFE, which had low success rates for forming bilayer lipid membranes or formed unstable bilayer lipid membranes.

Electrolyte solution ensures the matrix is electrically conductive and, on the application of electrical potentials, the measurement of currents is possible, which is useful in certain applications such as for measuring ion channel protein activity.

Preferably, hydration of the support matrix occurs prior to application of a lipid containing solution in order to aid in the successful formation of a bilayer lipid membrane.

Preferably, the hydration solution infiltrates the support matrix pores.

In one embodiment, infiltration is completed by immersion of the matrix in the hydration solution and subsequent use of pressure to force (positive pressure) or suck (negative pressure) the hydration solution into the support matrix. Other methods to infiltrate the hydration solution into the matrix include use of a centrifuge and/or ultrasonification.

The exact mechanism is not certain but the inventors understand that when the infiltration processes described are used, the aqueous electrolyte solution is drawn or forced into the pores of the support matrix. It is understood by the inventors that these treatments are important in overcoming the repulsive forces between the aqueous solution and the hydrophobic support matrix and ensure pore infiltration.

The exact composition of the aqueous electrolyte solution depends on the requirements of the application which the bilayer lipid membrane is to perform. For example, if the bilayer lipid membrane is to have a proteoliposome containing solution applied to insert a protein into the support matrix or bilayer lipid membrane (as described later in this specification) then the electrolyte's composition will depend on the functional protein contained with the applied solution.

In one embodiment, the aqueous electrolyte solution contains an ion to which the protein to be inserted responds to. Other components may also be added to allow effective function of the protein including (but not limited to) buffer solutions and other compounds important for protein function.

More specifically, in one embodiment where the protein is a sodium ion channel protein, the hydration solution includes:
300 mM NaCl (sodium chloride); and,
10 mM HEPES (N-2-Hydroxylethylpiperazine-N-2-ethane sulphonic acid) buffer adjusted to pH 7.4 with hydrochloric acid/potassium hydroxide.

In another embodiment, where the protein is a large conductance, calcium activated, voltage gated calcium ion channel protein, the hydration solution includes:
140 mM potassium hydroxide;
2 mM potassium chloride;
20 mM HEPES (N-2-Hydroxylethylpiperazine-N-2-ethane sulphonic acid) buffer adjusted to pH 7.2 with methanesulphonic acid;
5 mM HEDTA N(2-Hydroxyethyl)ethylenediaminetriacetic acid); and,
titrated to a concentration of 10 μM free Ca²⁺ with 0.1 molar calcium chloride as determined by a Ca²⁺ ion selective electrode.

It is the inventors' experience that preloading of functional proteins into a support matrix described below is assisted by application of an aqueous electrolyte resulting in the present invention being more efficient than the methods disclosed in the prior art. Therefore, when currents are measured across the support matrix after formation of a bilayer lipid membrane, higher currents are measured across these membranes. This indicates that there is a higher rate of association of functional proteins with the support matrix of the present invention. It should be appreciated that this may be advantageous in applications such as drug discovery or analytical purposes where low detection limits are required.

In a preferred embodiment, the lipid containing solution in step (c) is a phospholipid solution.

In one embodiment, the lipid solution includes phosphatidyl choline and cholesterol in n-octane.

By way of example, phosphatidyl choline may be prepared using the method of Singleton, W. S. et al (1965) and extracted from egg yolks.

In an alternative embodiment, the lipid solution is a mixture of phosphatidyl ethanolanine and phosphatidyl choline dissolved in n-decane.

In both of the above examples, the solutions formed are preferably centrifuged at 10,000 RPM for one minute and the supernatant is the phospholipid solution used to form the bilayer lipid membrane.

Preferably, lipid solution is applied to the outer surface of the support matrix. In a preferred embodiment, the lipid solution is applied by ‘painting’ the solution onto the support matrix. In an alternative embodiment the lipid solution is ‘folded’ as a monolayer of lipids onto the support matrix.

On application, the lipid solution forms a bilayer lipid membrane on the support matrix. In an alternative embodiment, steps (b) and (c) occur simultaneously where the hydration solution and lipid solution are the same or these solutions are mixed together.

In order to use the bilayer lipid membrane as a biosensor for example, protein is applied to the membrane. Given the delicate nature of proteins, these proteins are applied to the membrane in a protein containing solution.

The inventors envisage that most proteins may be used in the present invention. Preferred proteins include ion channel proteins. Preferably, these proteins include one or more of the following: alamethicin, BK ion channel proteins, and sodium (Na⁺) ion channel proteins. Other proteins also envisaged include hERG channel proteins and viral ion channel proteins.

In one embodiment, the protein containing solution is a lipid mixture that contains the protein and contains the bilayer lipid membrane forming solution. That is, the protein containing solution may also be the bilayer lipid membrane forming solution and steps (c) and (d) above occur simultaneously. This should not be seen as limiting as it should be appreciated that steps (c) and (d) could be completed separately using different solutions. The bilayer lipid membrane formed on the support matrix is itself is a potentially useful product for later use in various applications such as biosensors.

In one preferred embodiment, the protein containing solution is a proteoliposome solution. This solution may be added after a bilayer lipid membrane has been formed or, may be used as a combination hydration and protein containing solution, effectively completing hydration and protein loading steps simultaneously so the matrix is ready for membrane formation at a later stage (a ‘pre-loaded’ matrix).

Proteoliposomes are sub-microscopic vesicles of phospholipids of the kind that form bilayer lipid membranes. The proteoliposomes contain one or more functional proteins which are inserted into the bilayer lipid membrane and are a convenient method to both store the protein and allow it to be used in applications such as the present invention.

In one embodiment, a proteolipisome containing solution may be formed by:
(a) combining 50 mg of 1-palmitoyl-2-oleoyl-sn-glycero-3-phosphoethanoamine, 20 mg of 1-palmitoyl-2-oleoyl-sn-glycero-3-phospho-serine (sodium salt), 10 mg of phosphatidyl choline and 10 mg of cholesterol to form a lipid solution;
(b) dispersing the lipid solution of step (a) in 9 ml of a reconstitution buffer containing 15 mM HEPES, 0.5 mM EGTA, 300 mM NaCl and 200 mM of sucrose adjusted to pH 7.4 using 0.05M potassium hydroxide (KOH);
(c) sonicating the mixture of step (b) twice for 20 seconds and then chilling on ice;
(d) mixing the sonicated mixture of step (c) with 90 μl of detergent and 900 μl of a purified protein solution and then ice for 20 minutes;
(e) freezing and thawing the result of step (d) twice in a dry ice/ethanol bath before centrifuging for 30 minutes;
(f) recovering the pellet formed in the centrifuge during step (e) and re-suspending this in 900 μl of reconstitution buffer thereby forming the proteoliposome containing solution.

Preferably, before final use, the proteoliposome containing solution is thawed and sonicated for 10 seconds.

Preferably, the protein containing (proteoliposome) solution is added to the support matrix by immersion and a subsequent infiltration process. In one embodiment, the support matrix is in contact with the proteoliposome solution for a duration of approximately 120 minutes.

The result of this process of adding a protein is termed a ‘pre-loaded’ bilayer lipid membrane.

Reference to the term ‘preloaded’ should be understood to mean an association of a protein with a support matrix.

As mentioned above, alamethicin protein can also be used in accordance with the present invention. Alamethicin is a peptide that spontaneously inserts itself into a bilayer lipid membrane. Molecules of alamethicin can diffuse within the plane of the membrane and may associate with other alamethicin molecules to form a channel for the passage of small ions when a voltage is applied across a bilayer lipid membrane. In this embodiment, alamethicin is simply dissolved in ethanol and applied to the bilayer lipid membrane. The bilayer lipid membrane has been formed in a previous step (step (c)) using a lipid containing solution and the alamethicin is applied absent of lipid solution. In this case, the protein is still active and able to be ‘pre-loaded’.

In a further alternative, steps (b), (c) and (d) all occur simultaneously.

One surprising result that the inventors have found was that a support matrix pre-loaded using the methods described can be stored for an extended period of time (at least 80 days) and the protein remains associated and stable (active) with the matrix.

A further surprising result found by the inventors was that a pre-loaded support matrix does not require the protein to be reinserted, even after the bilayer lipid membrane has been rinsed away and re-formed.

For example, the support matrix may be rinsed with a solvent to remove the existing bilayer lipid membrane. In one embodiment, the support matrix is washed with 100% ethanol and subsequently further rinsed with water to remove the membrane. Other methods of washing the matrix may also be completed without departing form the scope of the invention. A new bilayer lipid membrane is then re-formed on the support matrix and proteins previously applied then populate the new membrane.

It is envisaged that the mechanism responsible for this is that, during exposure to the protein containing solution, either the proteoliposome compounds, if present, or the protein migrate into the interstices of the support matrix to a point where they are not removed by the rinsing procedure yet remain stable and active within the matrix. The above finding has considerable benefit as bilayer lipid membranes tend to be unstable as are proteins when not stabilised in some manner.

In this case, the membrane can be removed altogether for storage and transport and then re-formed at a later date without degrading the protein activity.

In the inventors' experience, the pre-loaded support matrix (rinsed or membrane containing) may be stored in a refrigerator at 4° C. for at least 80 days and still retain protein activity.

This is also advantageous as the user does not have to re-apply the delicate protein solution but rather, only has to complete the step of reforming the membrane (if this has been removed) which is simpler process and saves time.

This extended duration of stability and robustness lends itself to testing and diagnosis applications such as use in biosensors.

A further advantage of a pre-loaded support matrix found by the inventors is that when protein activity is measured and tested, the response measured is large and easy to measure which is particularly useful in testing proteins with small degrees of activity. The invention as described above relates to methods to form a bilayer lipid membrane and use of the membrane and/or matrix to stabilise and support protein activity. It results in the production of bilayer lipid membranes that are more robust and durable than standard planar bilayer lipid membranes produced by methods currently known in the literature.

The bilayer lipid membranes formed by the method described herein are amenable to the introduction of proteins such as ion channel proteins. The present invention allows for the development of bilayer lipid membranes with proteins inserted that are specific for a desired application such as testing the activity of specific ion channels.

Membrane receptors and ion channels function with a high sensitivity and selectivity for a wide range of analytes, with particular significance to medicine, environmental monitoring, biosecurity and drug discovery. The durability and robustness of the bilayer lipid membranes formed by the method described herein will potentially allow the development of biosensors that can be used under a variety of conditions and display beneficial characteristics of longevity and stability to allow successful development of this technology. It is envisaged that the preloaded support matrices formed by this method are of use in research applications such as the study of cell and protein processes and the replication of these as they would occur in vitro.

### BRIEF DESCRIPTION OF DRAWINGS

Further aspects of the present invention will become apparent from the following description which is given by way of example only and with reference to the accompanying drawings in which:

FIG. 1 shows a representation of a polystyrene cuvette acting as a fixture for the support matrix in one embodiment;

FIG. 2 shows a diagrammatic representation of a dual chamber arrangement acting as a fixture for the support matrix in an alternative embodiment;

FIG. 3 shows a diagrammatic representation of a dual tube arrangement acting as a fixture for the support matrix in an alternative embodiment;

FIG. 4 shows a graph of currents recorded over time across a bilayer lipid membrane containing alamethecin;

FIG. 5 shows a graph of currents recorded over time associated with BK ion channel proteins reconstituted in a reformed bilayer lipid membrane supported on a PTFE support matrix;

FIG. 6 shows a graph of currents recorded over time associated with sodium ion channel protein reconstituted in a bilayer lipid membrane;

FIG. 7 shows a graph of the effect of the application of tetrodotoxin on ion channel protein function for sodium ion channel proteins;

FIG. 8 shows a graph of currents recorded over time associated with ion channel currents produced in a reformed bilayer lipid membrane;

FIG. 9a shows the current across a bilayer lipid membrane immediately following reformation of the bilayer lipid membrane;

FIG. 9b shows the current across a bilayer lipid membrane 120 minutes following reformation of the bilayer lipid membrane;

FIG. 10a shows a graph of currents recorded over time associated with Na⁺ channel currents after a preloaded filter has been stored for 80 days before it has had tetrodotoxin applied to it;

FIG. 10b shows a graph of currents recorded over time associated with Na⁺ channel currents after a preloaded filter has been stored for 80 days after it has had tetrodotoxin applied to it;

FIG. 10c is a graphical representation of the mean currents present in FIGS. 10a and 10b;

FIG. 11a is a graphical representation of currents measured over time across a bilayer lipid membrane formed on a nylon support matrix before addition of veratridine;

FIG. 11b is a graphical representation of currents measured over time across a bilayer lipid membrane formed on a nylon support matrix after addition of veratridine;

FIG. 11c is a graphical representation of currents measured over time across a bilayer lipid membrane formed on a nylon support matrix after application of tetradotoxin;

FIG. 11d shows graphically the mean currents measured in FIGS. 11a to 11c; and,

FIG. 12 shows the mean resistance across a PTFE support matrix before and after hydration by centrifugation.

FIG. 13 shows the capacitance of a membrane formed on a matrix that had been centrifuged during hydration.

### BEST MODES FOR CARRYING OUT THE INVENTION

Examples are now described for support matrix selection, preparation of a bilayer lipid membrane, methods for inserting a proteoliposome into a support matrix, and examples showing the matrix in operation confirming the presence of a bilayer.

### Example 1

### Support Matrix Selection

A trial was completed to determine the characteristics of the ideal support material according to the method of the present invention. An ideal support matrix is one that forms a bilayer lipid membrane and in which functional protein activity can be observed. In the example, ion channel activity is used as an indicator of protein activity.

Six types of support matrix were tested with pore sizes roughly equivalent. The support matrices were preloaded using two types of method (vacuum infiltration and centrifugation) and subsequent success in forming membranes and channel activity measured. More discussion is provided below on exact methods to form the membrane in subsequent Examples.

To determine the ideal support matrix characteristics, two attributes (hydrophobicity and pore shape) were investigated, known to the inventors as being of high relevance in forming an effective and useful membrane.

Each support matrix material's hydrophobicity was determined using a droplet test. A two microlitre droplet of water was placed on the surface of the support matrix and the contact angle that the droplet formed with the matrix was used to give an indication of the matrix hydrophobicity; i.e., more hydrophobic materials have lower contact angles and less hydrophobic materials have high contact angles.

Pore shape refers to the shape of the pores in each support matrix material and the resistance they provide to the flow of fluid through the matrix. This is indicated by a bubble point where materials with a high bubble point have a higher resistance to the flow of fluids through the pores (and thereby more irregular/tortuous pore shapes) and vica versa.

The results found are summarised in Table 1 below.

TABLE 1

| | | | | | | Frequency |
|---|---|---|---|---|---|---|
| | | | | | | of |
| | | | | | | Observing |
| | | | | | Success in | Ion |
| Support | Pore | Bubble | Contact | Preloading | Forming | Channel |
| Material | Size | Point | Angle | Method | Membranes | Activity |
| PTFE | 5.0 μm | 0.70 | 43° | Vacuum | 100% | 100% |
| | | | | Infiltration | | |
| PTFE | 0.45 μm | 0.63 | — | Centrifugation | 100% | 100% |
| Nylon | 5.0 μm | 0.21 | 44° | Vacuum | 100% | 80% |
| | | | | Infiltration | | |
| Silanised Silver | 5.0 μm | Not | 80.5° | Vacuum | 100% | 17% |
| | | Detected | | Infiltration | | |
| Unsilanised | 5.0 μm | 0.14 | 120° | Vacuum | 0% | Not |
| Silver | | | | Infiltration | | Applicable |
| Polycarbonate | 5.0 μm | 0.08 | 98° | Vacuum | 100% | 0% |
| | | | | Infiltration | | |

As can be seen from Table 1, support matrix materials such as unsilanised silver and polycarbonate which have a low bubble point and high contact angle showed virtually no ability to be preloaded with functional ion proteins. However, materials with a high bubble point and a low contact angle such as PTFE and to a lesser degree nylon were found to successfully support bilayer lipid membranes and be preloaded with functional ion proteins by the method of the present invention.

Therefore, based on Example 1, preferred support matrix materials are characterized by high hydrophobicity (contact angle greater than 50°) and high resistance to flow through pores (bubble point greater than 0.20). The most preferred material to act as a support matrix was PTFE.

It should also be noted that some substances have a low contact angle and high bubble point. These materials showed different success rates and abilities to be preloaded with functional ion proteins. For example, nylon and PTFE have almost identical contact angles but the bubble number for PTFE is almost three and a half times that of nylon. The success rate for these substances of supporting bilayer lipid membranes formed by the present invention is 100%. However, the frequency which these materials can be successfully preloaded with functional ion proteins is 100% for PTFE and 80% for nylon. This implies that the more irregular and tortuous pores in the PTFE provide a more suitable environment for functional ion proteins to be preloaded into and to subsequently function in any bilayer lipid membrane formed therein.

The success rate of forming bilayer lipid membranes on silanised silver filters was 100% whereas that on unsilanised silver was 0%. This indicates that the hydrophobic nature of the pore is an important factor in the support matrix ability to support the formation of a bilayer lipid membrane according to the present invention.

### Example 2

### Support Matrix Preparation

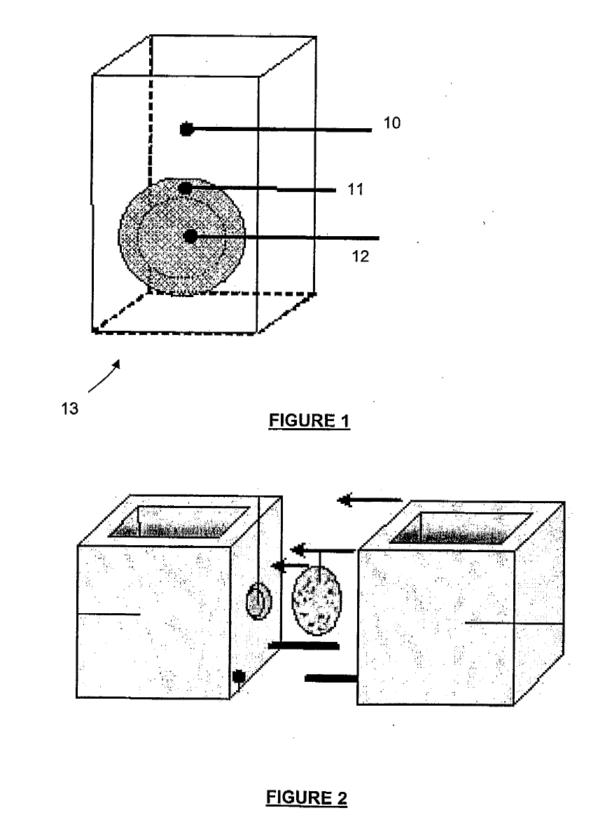

Referring to FIG. 1, a fixture (cuvette) 10 to hold a support matrix 11 was prepared by cutting down a commercially available polystyrene semi-micro cuvette to form a cuvette 10 with dimensions of approximately 10 mm wide×4 mm deep×45 mm high and a 1 mm wall thickness.

An approximately 1 mm diameter hole 12 was drilled in the front of the cuvette 10, located approximately 5 mm above the base of the cuvette 13. A piece of PTFE filter (support matrix 11) with a 5 μm porosity, cut into a circle of approximately 3 mm diameter, was placed over the hole 12 so that the hole 12 was overlapped on all sides. The support matrix 11 was then fastened to the cuvette by melting the polystyrene material around the matrix such that a firm seal was formed between the polystyrene cuvette and the matrix.

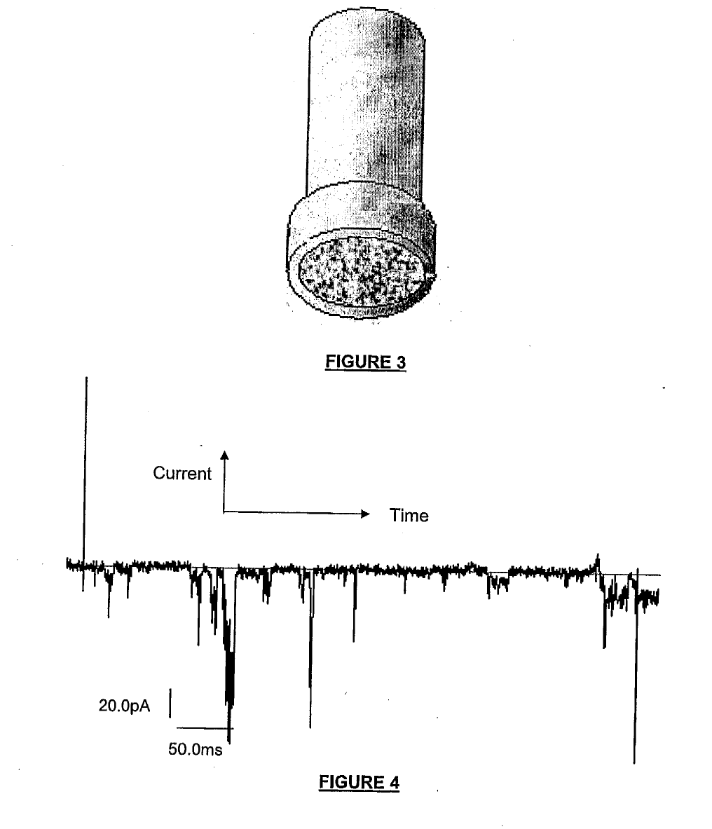

Alternative arrangements for securing the support matrix are shown in FIGS. 2 and 3. FIG. 2 shows a two box arrangement with the support matrix sealed between the two boxes. FIG. 3 shows an arrangement in a tube where the support matrix seals around the circumference of the tube at a tube collar point.

It should be appreciated from the above example, that the fixture can take various shapes with the proviso that the fixture needs to retain the support matrix and that the support matrix should form a seal around a hole or similar forcing liquid to pass through the matrix. Reference in further examples will be made to use of the cuvette of FIG. 1. This should not be seen as limiting.

### Example 3

### Support Matrix Hydration

The filters were hydrated by filling the cuvette 10 with an aqueous electrolyte solution. Once filled, the cuvette 10 was immersed in the same solution contained in a larger beaker.

Infiltration of the pores of the matrix was completed by placing the beaker containing the cuvette and hydration solution in a vacuum desiccator which was then evacuated with a water pump to 75 kPa. Following evacuation for approximately 120 minutes, the pressure in the desiccator was allowed to equilibrate with the atmosphere.

It is the inventors' experience that this step ensures that electrolyte overcomes the support matrix hydrophobic properties and fully infiltrates the support matrix pores.

### Example 4

### Bilayer Lipid Membrane Formation

A bilayer lipid membrane forming solution was prepared using 0.5% (w/w) of phosphatidyl choline extracted from egg yolks and 2% (w/w) cholesterol in n-octane was made before application by centrifuging the solution at 10,000 rpm for approximately 1 minute and collection of the supernatant. 10-20 μl of the bilayer lipid membrane forming solution was then applied to the outer surface of the support matrix using a 10 μl micro syringe. On application, the solution forms a bilayer lipid membrane on the support matrix.

### Example 5

### Proteoliposome Preparation

A proteoliposome solution was produced in preparation for loading proteins onto the membrane. The solution was produced by combining:
1. 50 mg of 1-palmitoyl-2-oleoyl-sn-glycero-3-phosphoethanoamine,
2. 20 mg of 1-palmitoyl-2-oleoyl-sn-glycero-3-phospho-serine (sodium salt),
3. 10 mg of phosphatidyl choline and,
4. 10 mg cholesterol.

The combined mixture was dispersed in 9 ml of a reconstitution buffer containing 15 mM HEPES, 0.5 mM EGTA, 300 mM NaCl and 200 mM of sucrose adjusted to pH 7.4 using 0.05M potassium hydroxide (KOH).

The lipid mixture was then sonicated twice for 20 seconds and then chilled on ice. Nine hundred microlitres (μl) of the lipid mixture was then mixed with 90 μl of detergent and 900 μl of a purified protein solution and then left on ice for 20 minutes.

The mixture was then left to freeze and thawed twice in a dry ice/ethanol bath before being centrifuged for 30 minutes. The pellet formed at the bottom of the centrifuged tube(s) was then re-suspended in 900 μl of reconstitution buffer to form the proteoliposome containing solution.

Before final use, the re-suspended proteoliposomes were frozen as aliquots at −80° C. and then thawed and sonicated for 10 seconds.

### Example 6

### Addition of Lipid Containing Solution

10-20 μl of proteoliposome solution was added to a beaker containing the support matrix 11 and cuvette 10 from Example 4. The beaker solution including proteoliposome was then stirred with a magnetic stirrer for 3-5 minutes.

### Example 7

### Preloading the Filters with Liposomes

Cuvettes were immersed in a suspension of proteoliposomes in a bath solution. The beaker was placed in a vacuum desiccator which was then evacuated with a water pump. Following evacuation for 120 minutes the pressure in the desiccator was allowed to equilibrate with the atmosphere.

### Example 8

### Confirmation of Membrane Formation

Alamethicin is a peptide that spontaneously inserts itself into a bilayer lipid membrane. Molecules of alamethicin can diffuse within the plane of the membrane and may associate with other alamethicin molecules to form a channel for the passage of small ions when a voltage is applied across a bilayer lipid membrane.

Given the above, alamethicin was used as an indicator of bilayer lipid membrane formation success.

In this example, alamethicin is dissolved in 100% ethanol (5 μg/ml) and stored at 4° C. After the bilayer lipid membrane is formed, alamethicin is added to solutions on both sides of the PTFE support matrix 11 to a final concentration of 100 ng/ml. Electrochemical measurements were then carried out using a two electrode system. Silver/silver chloride wires were used as the working and reference electrodes and based on measurements taken, channels were observed after 10 minutes.

The nature of the electrical current caused by the flow of ions is understood to be dependent on:
1. The concentration of ions bathing the bilayer lipid membrane,
2. The voltage applied and,
3. The concentration of alamethicin.

For the conditions used in the present invention the currents were as expected being brief spikes of varying magnitudes in the range of 10 to 200 pA. FIG. 4 shows an example graph of time (x-axis) versus current measured (y-axis) that is typical of the results obtained by the inventors in this example.

The results found conform to the pattern of results obtained by others in the prior art. Alamethicin will only form channels when it is in a single thickness of bilayer lipid membrane as the formation of functioning ion channels is limited by the need for the length of the alamethicin molecule to be greater than the width of the membrane it is contained in. Therefore, the observation of currents associated with alamethicin provides conclusive evidence for the formation on the filter material of a bilayer lipid membrane that creates a partition of high electrical resistance between the solutions on the inside and outside of the cuvette as in the present invention.

### Example 9

### Protein Insertion into the Bilayer Lipid Membrane

BK ion channels used in this example (also called large conductance, calcium activated, voltage gated potassium ion channels) are transmembrane proteins that have an important function in repolarising excitable cells following an excitation event. The functional ion channel is a tetramer of four identical subunits. The channel is activated by calcium and gated by positive electrical potentials and is selective for potassium ions. Proteoliposomes are a commonly used method for inserting ion channel proteins into planar lipid bilayers. The inventors used this technique to test whether functional BK channels were able to be inserted into the bilayer lipid membrane formed by the present invention as confirmed in Example 8 above.

Following the addition of proteoliposomes containing BK protein to the outside of the cuvette and stirring, voltage was applied between the inside and outside of the cuvette and the resulting currents observed.

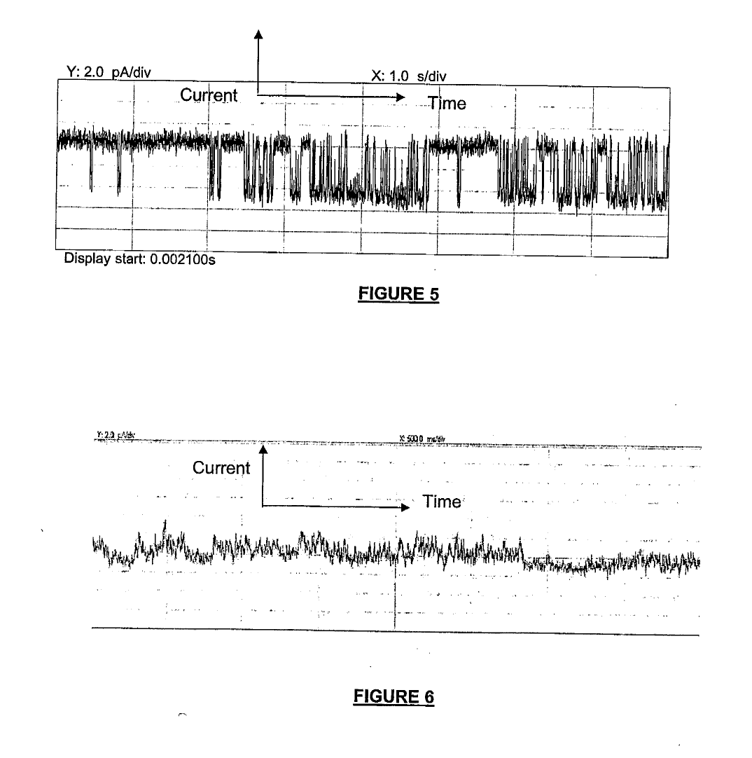

An example of the current profile found is shown in FIG. 5 which shows the rapid switching of current between two levels, representing the open and closed states of a single protein molecule. This confirms the presence of a functioning ion channel in the bilayer lipid membrane.

### Example 10

### Sodium Ion Channel Protein Insertion

Sodium (Na⁺) ion channels (voltage gated sodium ion channels) are another physiologically important integral membrane protein. These proteins cause the primary action in the generation of the current pulse in excitable cells and differ to BK channel proteins.

The inventors use proteoliposomes containing a Na⁺ channel protein to insert Na⁺ ion channels into filter supported bilayer lipid membranes produced by the present invention. These channels differ from BK channels in that they have a lower conductance and therefore produce smaller currents. Furthermore, they normally open for a few milli-seconds following the application of a voltage pulse which makes it difficult to record activity. To overcome the latter difficulty a pharmacological agent, veratridine, which causes Na⁺ channels to stay open longer when stimulated by a voltage pulse, was used in the preparations.

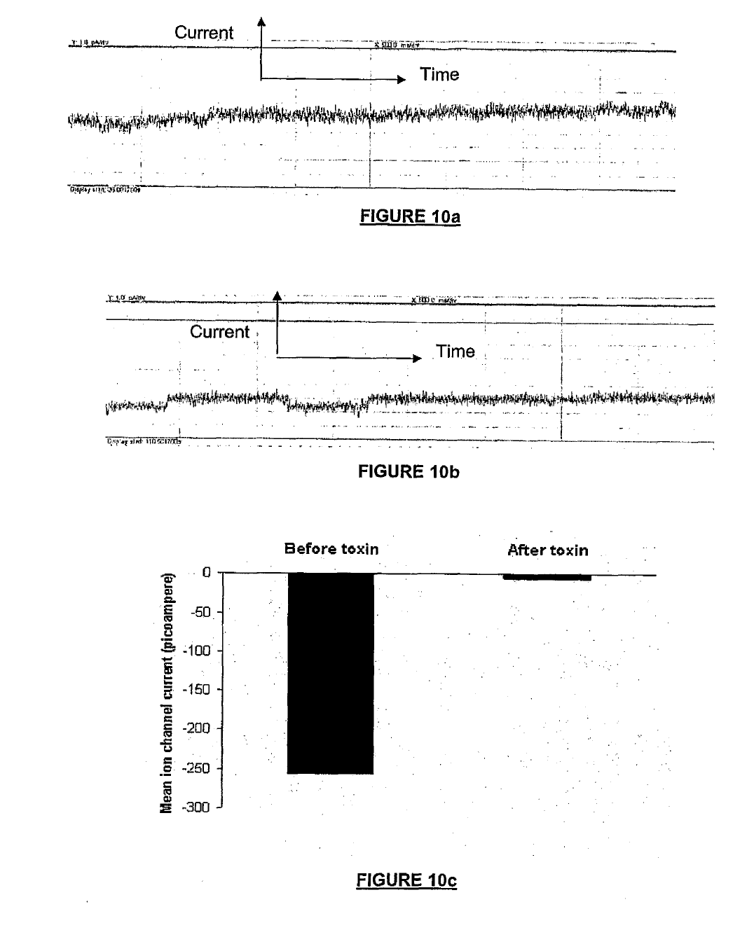

An example of the recordings obtained in this Example is shown in FIG. 6. The current transitions associated with the opening and closing of a single Na⁺ channel molecule are clearly seen. These traces indicate the function of multiple channels of activity. Note that an example of activation by veratridine of sodium channels using the method described is also shown in FIGS. 10a and 10b.

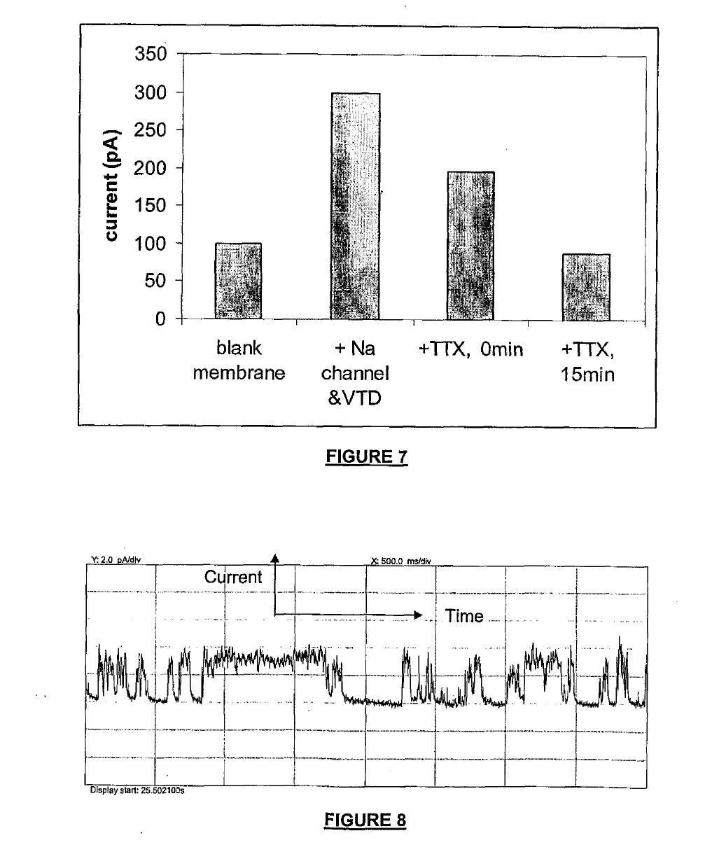

The response of the currents to stimulation by veratridine provides evidence that they are associated with Na⁺ channels and that the channels retain the ability to respond to this pharmacological agent. The ability of these channels in filter supported membranes to respond to pharmacologically active agents in the same way as they do in their native state in cells can be further analysed by testing for a response to the application of tetrodotoxin which blocks Na⁺ channels. FIG. 7 shows the currents for a sequence of measurements before and after the application of tetrodotoxin.

The measured current shown on the y-axis of the graph of FIG. 7 is the result of sodium ions flowing through the ion channel. FIG. 7 shows the increase in current from a membrane without a sodium ion channel (labelled ‘blank membrane’) to when sodium ion channels are added to the membrane (labelled ‘+Na Channel & VTD’). A reduction in current is seen shortly after the application of 200 μmolar tetrodotoxin and 15 minutes after the tetrotoxin application, the current decreased to the level measured for the blank membrane before the addition of the channel protein. This, together with the activation by veratridine, indicates that the protein, when inserted into the supported bilayer lipid membrane produced by the present invention, is able to respond to these compounds in a manner that mimics the native state.

### Example 11

### Bilayer Lipid Membrane Reformation

After the formation of a bilayer lipid membrane and insertion of functional protein into the membrane, the support matrix was rinsed with 100% ethanol, and subsequently rinsed three times with water purified by reverse osmosis. This effectively removes the existing bilayer lipid membrane and a new bilayer lipid membrane was then re-formed on the support matrix using the method described in Examples 1 to 4.

The above step was completed to determine if the support matrix could be re-used. A surprising result of the above trial was that, as the support and previous bilayer lipid membrane had been treated with proteoliposomes containing channel protein before rinsing, the reformed bilayer lipid membrane still exhibits channel activity without the addition of further proteoliposomes after rinsing. FIG. 8 shows an example of the currents observed for washed and re-formed bilayer lipid matrix.

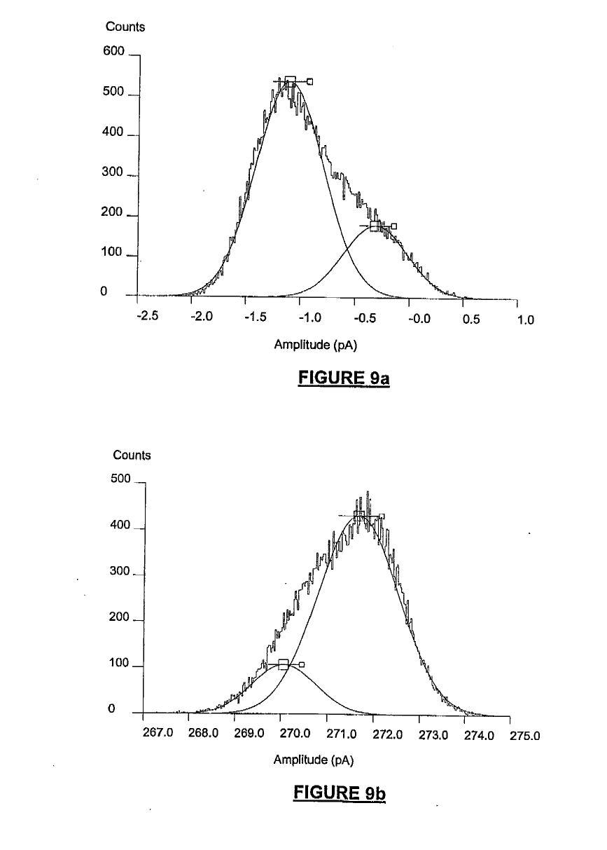

It is envisaged that the mechanism responsible for this is that, during exposure to the proteoliposomes either the proteoliposomes or the protein they contain migrates into the interstices of the support matrix to a point where they are not removed by the rinsing procedure. On formation of a new bilayer lipid membrane the proteins diffuse from the interior of the matrix and into the bilayer lipid membrane where their function is then observed. This understanding is supported by the observation that the currents observed increase with time as illustrated by the histograms in FIGS. 10a and 10b where the current immediately after the reformation of the bilayer lipid membrane is almost negligible at 1 pA (FIG. 9a) but 120 minutes later the current is significant at 271 pA (FIG. 9b).

### Example 12

### Protein Storage within a Support Matrix

As shown in Example 11, support matrices with bilayer lipid membranes containing functional proteins can be washed with 100% ethanol and rinsed with water purified by reverse osmosis.

A useful result from the fact that the proteins do not also wash out is that the support matrix has a degree of stability sufficient that the matrix may be stored in a refrigerator at 4° C. for varying periods of time for use when required.

It has been found that after storage extending over several weeks, reformation of a bilayer lipid membrane on these supports resulted in ion channel currents without a further exposure to proteoliposomes. FIG. 9 shows data for channels activated with vertridine in such a reformed bilayer lipid membrane, before and after the addition of the blocker tetrodotoxin (FIGS. 9a and 9b respectively). This support matrix was treated with proteoliposomes 80 days prior to reformation and testing. This extended duration is of importance in testing and diagnosis applications making it far easier to prepare and use such devices such as biosensors.

In the above example (shown in FIGS. 9a and 9b), the source of these currents was verified to be sodium ion channels by applying tetrodotoxin. Before the addition of tetrodotoxin the mean total current was 267 pA, with individual current transitions of approximately 5 pA. Following application of tetrodotoxin the mean total current decreased to 6.4 pA, with some small transitions of approximately 2 pA. FIG. 10c shows a comparison of the mean currents measured in FIGS. 10a and 10b. One skilled in the art will appreciate that although FIGS. 10a and 10b appear visually to be approximately the same, this is a limitation of the software used to present this data and the way it presents and modifies the y-axis. The actual mean currents measured are 267 pA in FIG. 10a and 60 pA in FIG. 10b.

The data demonstrates that storing the functional protein at 4° C. within the interstices of the support matrix provides conditions that allow the protein to retain its function for at least 11 weeks. In addition, preloading appears to enhance the current response observed.

In effect, the Example shows that the support matrix can be preloaded with functional proteins, stored for long periods of time and can then support a reformed bilayer lipid membrane. Washing the support matrix with a solvent with 100% ethanol does not appear to remove the functional proteins from the support matrix.

### Example 13

### Other Support Matrix Materials

Whilst as demonstrated in Example 1, PTFE is a preferred matrix, other materials with similar pore shape and hydrophobicity may also be used. By way of example, a nylon support was also tested by preloading the nylon support with proteoliposomes containing sodium ion channel proteins using the same methods described above.

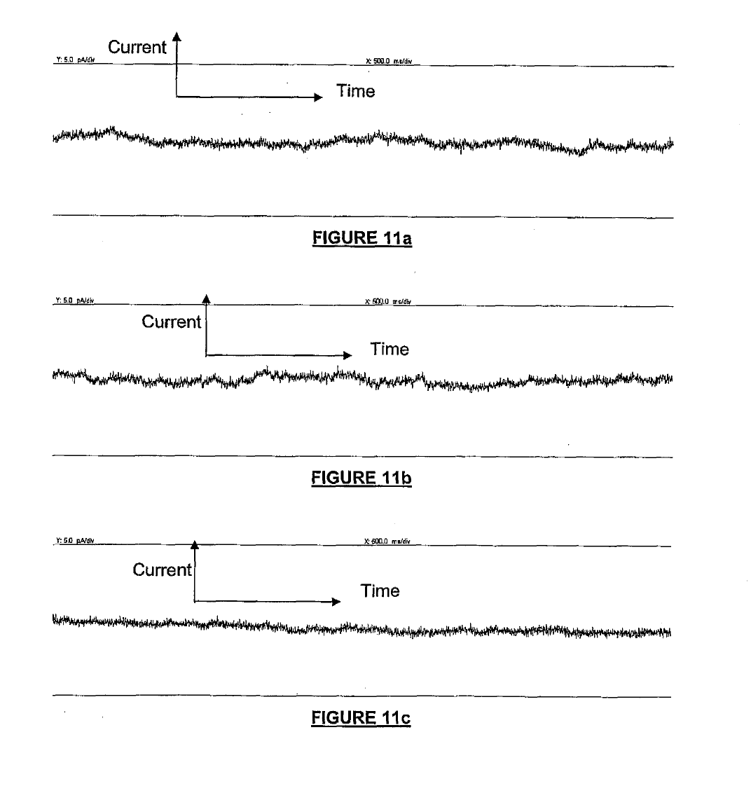

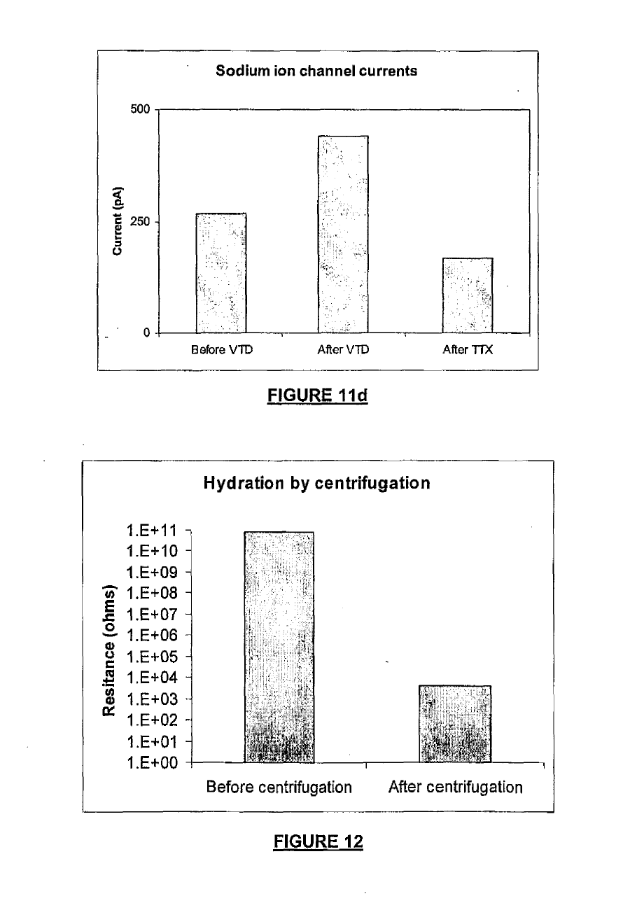

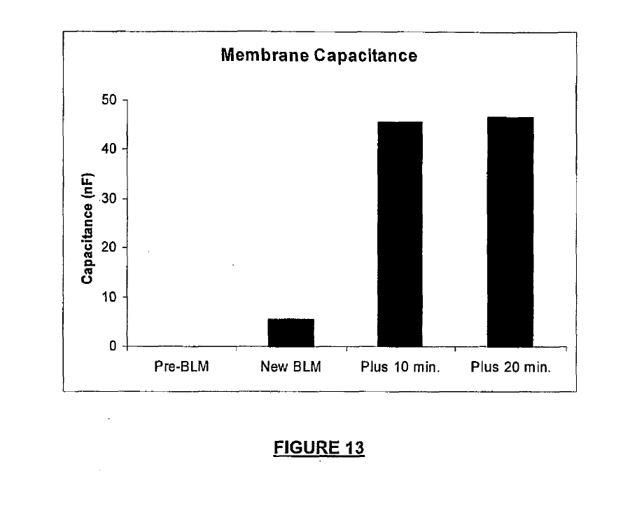

FIG. 11a shows the currents measured across a bilayer lipid membrane formed on a nylon support matrix and FIG. 11b shows currents measured after application of veratridine. FIG. 11c shows the current measured after addition of tetradotoxin. Referring to FIGS. 11a to 11c, it should be noted that, after application of veratridine, the current measured increases, while after application of tetradotoxin, there is less current observed. It is concluded by the inventors that the currents measured are a result of the bilayer lipid membrane and preloaded ion channels.

Note that the zero points in FIGS. 11a, 11b and 11c are shown so they appear to be on the same scale due to a lack of resolution in the y-axis. The true variation is more readily noticeable in FIG. 11d which summarises the mean currents measured before activation by the application of veratridine, following activation by veratridine, and subsequent to addition of tetradotoxin.

### Example 14

### Infiltration Using Centrifugation

In Example 3 the support matrix was hydrated using a vacuum. It should be appreciated that other methods of hydration may also be possible for example use of elevated pressure. As described above, of key importance is ensuring the hydration solution is fully infiltrated into the matrix pores.

In an alternative example, the use of centrifugation to cause infiltration was tested. A support matrix 11 was prepared and secured in place using a cuvette arrangement. The arrangement used was a single well cut from a multiwell plate with a PTFE base. An electrolyte hydration solution containing 300 millimoles of sodium chloride and 10 millimoles HEPES buffered to a pH of 7.4, was added to the interior of the cuvette or fixture retaining the support matrix. The matrix and cuvette were then centrifuged at 5000 rpm for 30 minutes.

The degree of hydration was then measured with the result shown in FIG. 13 where the electrical resistance measured across the PTFE support matrix is shown before and after hydration by centrifugation.

As can be seen from this Figure, the resistance after centrifugation is considerably less than that before and hence it is concluded that the electrolyte has infiltrated the pores of the matrix, hydrating it and creating an electrical contact between each side of the support matrix.

### Example 15

### Membrane Formation on a Centrifuged Matrix

A further experiment was conducted to confirm that a bilayer lipid membrane could be formed on support matrices centrifuged as in Example 14 above.

Following hydration and measurement of resistance as above, membrane forming solution was applied to the support matrix material and electrical impedance spectroscopy (EIS) performed using a Gamry model EIS300 electrical impedance spectrometer to determine the complex impedance. A model circuit was fitted to the impedance data in order to obtain values for the electrical capacitance and resistance of the membrane so formed. Measurements were made immediately after the formation of the membrane and repeated 10 and 20 minutes later. The results are shown in FIG. 13.

Electrical capacitance is commonly use in the field of bilayer lipid membrane research to indicate the presence of a bilayer lipid membrane. The capacitance arises from the thinness of these membranes, their high electrical resistance, and high dielectric strength.

The capacitance value measured in these test increased dramatically on formation of the membrane and continued to increase over time. The increase observed is consistent with membrane formation spreading across the surface of the filter over time.

### Example 16

### Alternative Phospholipid Solutions

As described in Example 4, the bilayer lipid membrane can be formed using a lipid solution. In an alternative trial the inventors used a mixture of phosphatidyl ethanolanine and phosphatidyl choline in a ratio of 8:2 dissolved in n-decane at a total concentration of 50 milligrams per ml to form the lipid solution.

Currents measured across a bilayer lipid membrane formed using this alternative solution showed similar results and therefore that the solution can be varied without departing from the scope of the invention as described in at least Example 4.

It should be appreciated from the above Examples that the inventors have devised methods to create stable bilayer lipid membranes which can be used to measure ion channel activity and potentially other protein activity. One application envisaged by the inventors is use in biosensor applications. Of note was the surprising result that the preloaded support matrix can be washed and re-used without need to add further protein containing solution. This is of considerable benefit in commercial applications as the time to prepare such devices is significantly reduced and the device may be stored for significant periods of time and still produce desired results.

Aspects of the present invention have been described by way of example only and it should be appreciated that modifications and additions may be made thereto without departing from the scope thereof.

1. A method of preparing a bilayer lipid membrane to which at least one receptor protein may be applied, including the steps of:
a) preparing a porous and hydrophobic support matrix;
b) infiltrating the support matrix with an aqueous hydrating solution; and,
c) applying a lipid containing solution to the infiltrated support matrix which forms a bilayer lipid membrane on the support matrix.

2. A method of preparing a bilayer lipid membrane loaded with at least one receptor protein, including the steps of:
a) preparing a porous and hydrophobic support matrix;
b) infiltrating the support matrix with an aqueous hydrating solution;
c) applying a lipid containing solution to the infiltrated support matrix which forms a bilayer lipid membrane on the support matrix; and,
d) applying a receptor protein containing solution to the infiltrated support matrix of step (c).

3. A method of preparing a pre-loaded receptor protein containing support matrix, including the steps of:
a) preparing a porous and hydrophobic support matrix;
b) infiltrating the support matrix with an aqueous hydrating solution;
c) applying a receptor protein containing aqueous solution to the infiltrated support matrix of step (b) wherein the receptor protein or proteins are loaded within a bilayer lipid membrane located on the support matrix.

4. A method of preparing a pre-loaded receptor protein containing support matrix, including the steps of:
a) preparing a porous and hydrophobic support matrix;
b) infiltrating the support matrix with an aqueous hydrating solution;
c) applying a lipid containing solution to the infiltrated support matrix which forms a bilayer lipid membrane on the support matrix;
d) applying a receptor protein containing aqueous solution to the infiltrated support matrix of step (c); and;
e) washing the bilayer lipid membrane from the support matrix.

5. The method as claimed in claim 1 wherein the support matrix is a material characterised by having a high hydrophobicity.

6. The method as claimed in claim 1 wherein the hydrophobicity of the matrix material corresponds to a contact angle less than 50°.

7. The method as claimed in claim 1 wherein the support matrix is a material characterised by having pore shape that results in a high resistance to flow through the pores.

8. The method as claimed in claim 7 wherein the pore shape of the matrix material corresponds to a bubble point greater than 0.20.

9. The method as claimed in claim 1 wherein the support matrix material is polytetrafluoroethylene (PTFE).

10. The method as claimed in claim 1 wherein the support matrix material is prepared in step (a) by attaching the material to a fixture that holds the matrix in place and directs flow of a liquid through the support matrix.

11. The method as claimed in claim 1 wherein the aqueous hydrating solution is an electrolyte solution.

12. The method as claimed in claim 1 wherein infiltration of the support matrix in step (b) occurs prior to application of a lipid containing solution.

13. The method as claimed in claim 1 wherein infiltration results in aqueous hydrating solution passing into pores within the support matrix

14. The method as claimed in claim 1 wherein infiltration is completed by immersion of the matrix into the aqueous hydrating solution and subsequent forcing of the solution into the matrix using methods selected from: pressure (positive pressure), vacuum (negative pressure), centrifuge and/or ultrasonification.

15. The method as claimed in claim 1 wherein the aqueous hydrating solution contains an ion corresponding to a receptor protein to be added to the matrix.

16. The method as claimed in claim 1 wherein the aqueous hydrating solution contains at least one buffer solution.

17. The method as claimed in claim 1 wherein the receptor protein or proteins are in a lipid containing solution which is a phospholipid solution.

18. The method as claimed in claim 1 wherein the lipid containing solution includes phosphatidyl choline and cholesterol in n-octane.

19. The method as claimed in claim 1 wherein the lipid containing solution is a mixture of phosphatidyl ethanolanine and phosphatidyl choline dissolved in n-decane.

20. The method as claimed in claim 1 wherein the lipid containing solution is applied to the outer surface of the support matrix.

21. The method as claimed in claim 1 wherein the lipid containing solution also acts as an aqueous hydrating solution and infiltration and membrane formation occur simultaneously.

22. The method as claimed in claim 1 wherein the receptor protein or proteins are functional.

23. The method as claimed in claim 1 wherein the receptor protein or proteins are integral membrane receptor proteins.

24. The method as claimed in claim 1 wherein the receptor protein or proteins are ion channel proteins.

25. The method as claimed in claim 1 wherein the receptor protein or proteins are selected from one or more of: alamethicin, BK ion channel proteins, sodium (Na⁺) ion channel proteins, hERG channel proteins and viral ion channel proteins.

26. The method as claimed in claim 1 wherein the receptor protein containing solution is a proteoliposome solution.

27. The method as claimed in claim 1 wherein the receptor protein containing solution also acts as a lipid containing solution and membrane formation and protein addition occurs simultaneously.

28. The method as claimed in claim 1 wherein the receptor protein containing solution is added to the support matrix by immersion and a subsequent infiltration step or steps.

29. The method as claimed in claim 1 wherein the receptor protein or proteins are in a solution containing alamethicin peptide dissolved in ethanol.

30. The method as claimed in claim 1 wherein infiltration, membrane formation and receptor protein addition occur simultaneously.

31. The method as claimed in claim 1 wherein the support matrix after membrane formation can be stored for an extended period of time and the bilayer lipid membrane remains associated and stable.

32. The method as claimed in claim 2 wherein the support matrix produced can be stored for an extended period of time and the receptor proteins remain associated and stable.

33. The method as claimed in claim 3 wherein the support matrix produced can be stored for an extended period of time and the receptor proteins remain associated and retain activity and, on formation or re-formation of a bilayer lipid membrane, the receptor proteins previously applied populate the newly formed bilayer lipid membrane.

34. The method as claimed in claim 4 wherein the support matrix bilayer lipid membrane is removed by washing the support matrix with 100% ethanol and subsequently further rinsing with water.

35. The method as claimed in claim 31 wherein the duration of stability is at least 80 days without retained receptor protein activity when the matrix is stored at refrigerated conditions.

36. A bilayer lipid membrane produced by the method as claimed in claim 1.

37. A support matrix produced by the method as claimed in claim 3.

38. A porous and hydrophobic support matrix pre-loaded with at least one receptor protein wherein the receptor protein or proteins are loaded within a bilayer lipid membrane.

39. The support matrix of claim 38 wherein the pre-loaded support matrix retains receptor protein activity in a stable form when stored at refrigerated conditions for a time period of at least 80 days.

40. The support matrix as claimed in claim 38 wherein the support matrix is a material characterised by having a high hydrophobicity.

41. The support matrix as claimed in claim 38 wherein the hydrophobicity of the matrix material corresponds to a contact angle less than 50°.

42. The support matrix as claimed in claim 38 wherein the support matrix is a material characterised by having pore shape that results in a high resistance to flow through the pores.

43. The support matrix as claimed in claim 42 wherein the pore shape of the matrix material corresponds to a bubble point greater than 0.20.

44. The support matrix as claimed in claim 38 wherein the support matrix material is polytetrafluoroethylene (PTFE).

45. The support matrix as claimed in claim 38 wherein the receptor protein or proteins are functional.

46. The support matrix as claimed in claim 38 wherein the receptor protein or proteins are integral membrane receptor proteins.

47. The support matrix as claimed in claim 38 wherein the receptor protein or proteins are ion channel proteins.

48. The support matrix as claimed in claim 38 wherein the receptor protein or proteins are selected from: alamethicin, BK ion channel proteins, and sodium (Na⁺) ion channel proteins.

49. The support matrix as claimed in claim 38 wherein the matrix supports formation of a bilayer lipid membrane.

50. The support matrix as claimed in claim 38 wherein the pre-loaded support matrix can be washed to remove a bilayer lipid membrane and be re-used to form a new membrane with pre-loaded receptor protein re-inserting into the new membrane.

51. A bilayer lipid membrane including at least one receptor protein located within the pores of a porous hydrophobic support matrix.

52. The bilayer lipid membrane as claimed in claim 51 wherein the bilayer lipid membrane retains receptor protein activity in a stable form when stored at refrigerated conditions for a time period of at least 80 days.

53. The bilayer lipid membrane as claimed in claim 51 wherein the support matrix is a material characterised by having a high hydrophobicity.

54. The bilayer lipid membrane as claimed in claim 51 wherein the hydrophobicity of the matrix material corresponds to a contact angle less than 50°.

55. The bilayer lipid membrane as claimed in claim 51 wherein the support matrix is a material characterised by having pore shape that results in a high resistance to flow through the pores.

56. The bilayer lipid membrane as claimed in claim 55 wherein the pore shape of the matrix material corresponds to a bubble point greater than 0.20.

57. The bilayer lipid membrane as claimed in claim 51 wherein the support matrix material is polytetrafluoroethylene (PTFE).

58. The bilayer lipid membrane as claimed in claim 51 wherein the receptor protein or proteins are functional.

59. The bilayer lipid membrane as claimed in claim 51 wherein the receptor protein or proteins are integral membrane receptor proteins.

60. The bilayer lipid membrane as claimed in claim 51 wherein the receptor protein or proteins are ion channel proteins.

61. The bilayer lipid membrane as claimed in claim 51 wherein the receptor protein or proteins are one or more of proteins selected from: alamethicin, BK ion channel proteins, and sodium (Na⁺) ion channel proteins.

62. The bilayer lipid membrane as claimed in claim 51 wherein the bilayer lipid membrane is formed from a lipid containing solution.

63. The bilayer lipid membrane as claimed in claim 62 wherein the lipid containing solution is a phospholipid solution.
